(19) United States
(12) Patent Application Publication
(10) Pub. No.: US 2025/0196022 A1
(43) Pub. Date: Jun. 19, 2025

(54) METHOD AND APPARATUS FOR PROCESSING BIRCH BARK

(71) Applicant: Innomost Oy, Kokkola (FI)

(72) Inventor: Sami Selkälä, Kokkola (FI)

(73) Assignee: Innomost Oy, Kokkola (FI)

(21) Appl. No.: 18/839,809

(22) PCT Filed: Feb. 23, 2023
(86) PCT No.: PCT/FI2023/050106

§ 371 Date: Aug. 20, 2024

(30) Foreign Application Priority Data
Feb. 24, 2022 (FI) 20225169

(51) Int. Cl. B01D 11/02; B03B 9/00; C07J 53/00; C08H 8/00; C10B 53/02

(52) U.S. Cl. CPC B01D 11/0288; B03B 9/00; C07J 53/002; C08H 8/00; C10B 53/02

(57) ABSTRACT

The present disclosure relates to a method for processing birch bark. The present disclosure further relates to an arrangement for processing birch bark.

### TECHNICAL FIELD

The present disclosure relates to a method for processing birch bark. The present disclosure further relates to an arrangement for processing birch bark.

### BACKGROUND

Birch (Betula spp.) is widespread throughout the Northern Hemisphere and harvested in huge volumes. Birch bark is produced in considerable quantities as a byproduct of the forest industry and its upgrading is almost totally neglected. Currently, this low-value side stream is burnt for combined heat and power production.

Birch bark could, however, find more valuable uses, for example as an additive in plastic composite materials. Birch bark consists of brown inner bark ˜75% and white outer bark ˜25%. The inner bark consists mainly of wood-like material such as lignin, pentosans and hexosans. The outer bark contains, by dry weight, up to 40% fats, fatty acids, resins and triterpenes, in particular betulin, at up to 30%. In addition, the outer bark contains up to 35% suberin. Valorization and upgrading of these compounds by using modern chemical technology opens up entirely new opportunities to produce new specialty chemicals from this low-value biomass stream.

It has been estimated that a pulp mill with an annual production capacity of 200,000 tonnes of birch kraft pulp produces enough bark to produce around 2,500 tonnes of betulin of around 95% purity and 4,000 tonnes of suberin acids per annum. Suberin polyester can be hydrolyzed by base treatment to multifunctional suberin acids, which are potential raw materials for paints, adhesives, lubricants and surface-active agents. New potential applications for betulin or betulin derivatives include pharmaceuticals and cosmetic products as well as agrochemicals.

### SUMMARY

A method for processing birch bark is disclosed. The method may comprise:
providing a raw material comprising birch bark,
screening the raw material comprising birch bark to remove fines to form a screened raw material,
separating the screened raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder.

An arrangement for processing birch bark is also disclosed.

The arrangement may comprise:
a first drum screening device,
a sorting device,
a drying device,
a first cutting mill,
a second drum screening device, and
a second cutting mill.

The arrangement may comprise
a first drum screening device,
a sorting device,
a drying device,
a first cutting mill,
a second drum screening device, and
a second cutting mill, arranged in the above specified order.

### BRIEF DESCRIPTION OF THE DRAWINGS

The accompanying drawing, which is included to provide a further understanding of the embodiments and constitute a part of this specification, illustrates various embodiments. In the drawings:

FIG. 1 presents a flow chart of one embodiment of the method for processing birch bark.

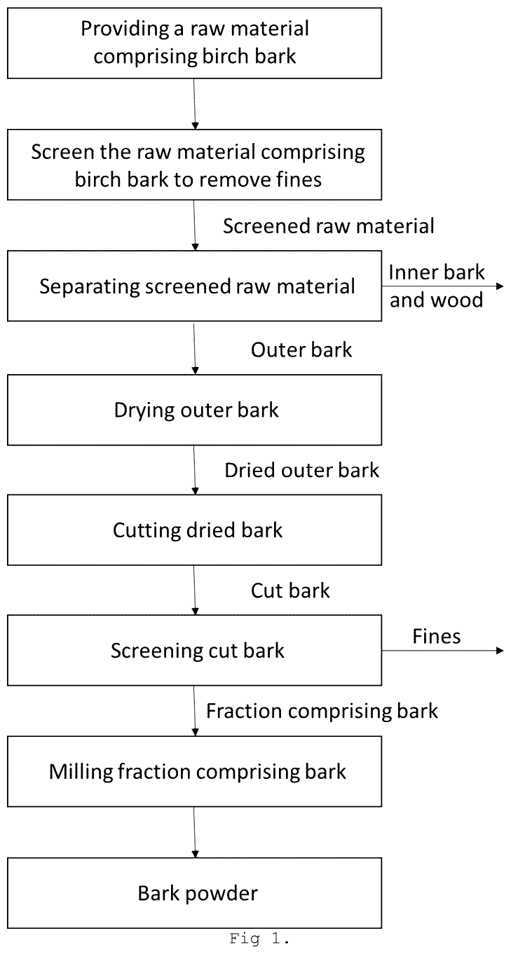

### DETAILED DESCRIPTION

Birch bark can broadly be said to consist of two distinct layers: an inner dark layer and an outer light layer. While the inner layer is wood-like and generally burned for energy. The outer, light or white, layer has been found to contain several different extractives that have different potential uses if extracted from the bark.

A method for processing birch bark is disclosed. The method may comprise:
providing a raw material comprising birch bark,
screening the raw material comprising birch bark to remove fines to form a screened raw material,
separating the screened raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder.

Birch bark may be acquired e.g. as side products or wastes from other processes of forest or paper industry. E.g. wood intended for the production of paper is commonly debarked to form debarked logs and a fraction containing a mixture of bark particles and wood removed from the logs in the debarking. If the raw logs are to be sawed to be used as e.g. construction material, the outermost parts including the bark layers is commonly discarded and may in some cases be burned for energy.

When the logs are debarked, the bark is separated from the logs to form a mixture of particles of various size that include both outer bark, inner bark, and some wood that has been separated from the logs. In the mixture of particles some of the outer bark, inner bark, and wood will be separate particles and some will be separated into the various components.

In one embodiment, screening the raw material comprising birch bark comprises screening the raw material comprising birch bark in a drum screening to remove particles with a particle size of 45 mm or less. In certain embodiments, screening the raw material comprising birch bark comprises screening the raw material comprising birch bark in a drum screening to remove particles with a particle size of 45 mm or less, or 40 mm or less, or 35 mm or less, or 30 mm or less, or 25 mm or less.

In one embodiment, separating the screened raw material into a fraction comprising inner bark and wood particles, and a fraction comprising outer bark comprises sorting the screened raw material optically to separate inner bark and wood particles from outer bark.

The inner bark after sorting also comprises moisture from the original raw material. In order to improve the efficiency of the extraction, the bark material fed into the extraction should contain as little moisture as possible.

In one embodiment, drying the fraction comprising outer bark comprises subjecting it to at least one drying using warm air.

The efficiency of the extraction is also dependent on the particle size of the solid material on which the extraction is performed. When the particle size is reduced, the relative surface area is increased which improves the efficiency of the extraction.

In certain embodiments, the dried bark is milled in a cutting mill to a particle size of 6 mm-45 mm. In certain embodiments, the dried bark is milled in a cutting mill to a particle size of approximately 6 mm-45 mm, or approximately 6 mm-40 mm, or approximately 6 mm-35 mm, or approximately 6 mm-25 mm, or approximately 6 mm-20 mm. In one embodiment, the dried bark is milled in a cutting mill to a particle size of approximately 45 mm or less.

By milling the dried bark in a cutting mill, it is possible to cut, mill, or grind the inner bark still attached to the outer bark into small particles that are separated from the outer bark. Once dried, the inner bark becomes brittle and is thus ground into fine particles in the milling. As the brittle inner bark is ground into finer particles than the outer bark, it is possible to separate the inner bark particles from the outer bark by screening the cut bark.

As will be obvious to a person skilled in the art, cutting or milling dried bark to a given size will produce a mixture of particles of various sizes. While the largest particles present in the resulting mixture will have the desired size, the mixture will inevitably also comprise particles of smaller size. As a non-limiting example, milling dried bark in a cutting mill to a particle size of approximately 45 mm may produce a mixture of particles in a size range of approximately 6 mm-45 mm.

In one embodiment, the cut bark is screened to remove particles of a size of approximately 6 mm or smaller. In certain embodiments, the cut bark is screened to remove particles of a size of approximately 6 mm or smaller, or approximately 5 mm or smaller, or approximately 4 mm or smaller, or approximately 3 mm or smaller.

In one embodiment, the fraction comprising bark is milled to a particle size of 0 mm-6 mm. In certain embodiments, the fraction comprising bark is milled to a particle size of 0 mm-6 mm, or 0 mm-5 mm, or 0 mm-4 mm, or 0 mm-3 mm, or 0 mm-2.5 mm, or 0 mm-2 mm, or 0 mm-1.5 mm, or 0 mm-1.0 mm, or 0 mm-0.5 mm.

Once the bark material has been processed into particle with a suitable particle size, the bark powder may be subjected to extractions and other chemical treatments to obtain chemical compounds and products for further use.

In one embodiment, the method further comprises subjecting the bark powder to a liquid-solid extraction to recover soluble components from the bark powder.

In certain embodiments, various chemical compounds may be extracted from birch bark powder directly using liquid-solid extraction using a suitable solvent. The solvent and the extraction process may be selected according to the compound to be extracted.

In one embodiment, the method comprises recovering betulin and/or suberin from the bark powder.

In one embodiment, the method comprises subjecting the bark powder to a liquid-solid extraction to recover betulin or suberin from the bark powder.

In one embodiment, the method further comprises subjecting the bark powder to a liquid-solid extraction to recover betulin from the bark powder.

In one embodiment, the method for extracting betulin from the bark powder comprises extracting the bark powder with an organic solvent under reflux conditions followed by isolation of the betulin by consecutive decantation and reslurrying steps. After removal of excess solvent, betulin may be collected.

In certain embodiments, the method for extracting betulin from the bark powder comprises extracting the bark powder with a solvent selected from methanol, ethanol, 1-propanol, isopropanol, 1-butanol, 2-butanol, 1-pentanol, 2-pentanol, 1-hexanol, methyl acetate, ethyl acetate, propyl acetate, isopropyl acetate, butyl acetate, cyclohexane, methyl cyclohexane, toluene, tetrahydrofuran, 2-methyltetrahydrofuran, methyl ethyl ketone, acetone, 2,2,5,5-tetramethyloxolane, 2-methoxyethanol, or any combination thereof under reflux conditions followed by isolation of the betulin by consecutive decantation and reslurrying steps. After removal of excess solvent, betulin may be collected.

In one embodiment, the method for extracting betulin from the bark powder comprises extracting the bark powder with isopropanol under reflux conditions followed by isolation of the betulin by consecutive decantation and reslurrying steps. After removal of excess solvent, betulin may be collected.

In certain embodiments, the purity of the betulin collected after the extraction may be at least 80%, or at least 83%, or at least 85%, or at least 88%, or at least 90%, or at least 93%.

In one embodiment, the method further comprises subjecting the bark powder to a liquid-solid extraction to recover suberin from the bark powder.

In one embodiment, the liquid-solid extraction to recover suberin comprises extraction using sodium isopropanoate followed by isolation of the suberin. As used herein, “sodium isopropanoate” refers to the sodium salt of isopropanol.

In certain embodiments, the method for extracting suberin from the bark powder comprises extracting the bark powder with sodium or potassium alkoxylate, or any combination thereof under reflux conditions followed by acidifying the solution of suberin salt to obtain an emulsion of suberin in water. After removal of excess water, suberin may be collected.

In certain embodiments, the method for extracting suberin from the bark powder comprises extracting the bark powder with sodium methoxylate, sodium ethoxylate, sodium propanoate, sodium isopropanoate, sodium butanoate, potassium isopropanoate, potassium propanoate, potassium ethoxylate, potassium butanoate, or any combination thereof under reflux conditions followed by acidifying the solution of suberin salt to obtain an emulsion of suberin in water. After removal of excess water, suberin may be collected.

In one embodiment, the method for extracting suberin from the bark powder comprises extracting the bark powder with sodium isopropanoate under reflux conditions followed by acidifying the solution of suberin sodium salt to obtain an emulsion of suberin in water. After removal of excess water, suberin may be collected.

In certain embodiments, the purity of the collected suberin may be at least 60% of 10-epoxy-18-hydroxyoctanedecanoic acid.

As used herein, “suberin” refers to complex polyester biopolymers that are lipophilic and composed of long chain fatty acids called suberin acids, and glycerol. The exact qualitative and quantitative composition of suberin monomers varies in different species. Some common aliphatic monomers include α-hydroxyacids (mainly 18-hydroxyoctadec-9-enoic acid) and α,ω-diacids (mainly octadec-9-ene-1,18-dioic acid). The monomers of the polyaromatics are hydroxycinnamic acids and derivatives, such as feruloyltyramine. Other examples of components included under in suberin are, 10-epoxy-18-hydroxyoctadecanoic acid and 9,10, 18-trihydroxyoctadecanoic acid

In one embodiment, the liquid-solid extraction to recover betulin comprises extraction with isopropanol followed by isolation of the betulin.

In certain embodiments, extracting chemical components from the birch bark powder may require additional steps in addition to extraction to get the chemical compounds in a sufficiently pure form.

In certain embodiments, other chemical components may be isolated from the bark powder either directly or by utilizing further chemical processes.

Once the chemical components of interest have been extracted from the bark powder, the remaining material may still be utilized to provide additional value. The bark powder may e.g. be burned for energy or used as filler material on several applications. The bark powder may also be combined with one or more similar materials before continued utilization.

In one embodiment, the bark powder is carbonized to form birch charcoal powder.

An arrangement for processing birch bark is disclosed herein.

The arrangement may comprise
a first drum screening device,
a sorting device,
a drying device,
a first cutting mill,
a second drum screening device, and
a second cutting mill.

The arrangement may comprise
a first drum screening device,
a sorting device,
a drying device,
a first cutting mill,
a second drum screening device, and
a second cutting mill, arranged in the above specified order.

Processing the birch bark in an arrangement according to the present disclosure enables separating the outer bark from the other materials included in a mixture of bark and wood material. By performing the separation using the arrangement of the present disclosure the material is processed in several steps to reach a successively higher fraction of outer bark in each of the steps while simultaneously lowering the total moisture content of the material.

In one embodiment, the first drum screening device removes particles of size of 45 mm or less and the second drum screening device removes particles of a size of 6 mm or less.

In certain embodiments, the first drum screening removes particles with a particle size of 45 mm or less, or 40 mm or less, or 35 mm or less, or 30 mm or less, or 25 mm or less. In certain embodiments, second drum screening removes particles of a size of approximately 6 mm or smaller, or approximately 5 mm or smaller, or approximately 4 mm or smaller, or approximately 3 mm or smaller.

In one embodiment, the sorting device is an optical sorting device.

In one embodiment, the drying device is a heated air drying device.

In one embodiment, the first cutting mill cuts the material to a particle size of approximately 45 mm and the second cutting mill cuts the material to a particle size of approximately 0 mm-6 mm.

In certain embodiments, the first cutting mill cuts the material to a particle size of approximately 45 mm, or approximately 40 mm, or approximately 35 mm, or approximately 25 mm, or approximately 20 mm. In certain embodiments, the second cutting mill cuts the material to a particle size of approximately 0 mm-6 mm, or 0 mm-5 mm, or 0 mm-4 mm, or 0 mm-3 mm, or 0 mm-2.5 mm, or 0 mm-2 mm, or 0 mm-1.5 mm, or 0 mm-1.0 mm, or 0 mm-0.5 mm.

The method for processing birch bark described in the current specification has the added utility of reducing the total amount of energy required to obtain a birch bark powder consisting of essentially only outer bark. Removing the inner bark and wood usually included in bark fractions, the amount of moisture is included in the bark is reduced significantly, meaning that less energy is required for drying the bark.

An additional advantage is that as the outer bark is removed from the bark fraction, the amount of material fed into the liquid-solid extraction may be reduced significantly, providing savings in energy and reducing the amount of solvent required for the extraction itself. A still further advantage of the methods disclosed herein is that all solvents used can be recycled and reused in the process, thereby lower the total amounts of harmful chemicals needed.

The arrangement for processing birch bark described in the current specification has the added utility of reducing the total amount of energy required to obtain a birch bark powder consisting of essentially only outer bark. Using the arrangement of the present disclosure, it is possible to obtain a dry powder consisting mainly of outer birch bark by a relatively low number of process steps. Removing excess or unwanted wet material before drying the material significantly lowers the total amount of energy needed for the whole process.

### Examples

Reference will now be made in detail to various embodiments.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

### Example 1-Processing Bark

Approximately 550 kg (2.2 m3) of birch bark material was acquired from a veneer factory. The crude birch bark mixture included about 54 weight-% or 297 kg of dry matter. The crude birch bark material was screened using a drum screening to remove particles with a size of 25 mm or less. Approximately 176 kg of material with a particle size of 25 mm or less was collected and discarded. Approximately 374 kg of wet material with a particle size of over 25 mm was collected. The wet material collected included both wood and bark pieces, including both inner and outer bark.

The collected wet material with a particle size of 25 mm or more was sorted using an optical sorting device to separate the outer bark from the inner bark and wood particles. Approximately 208 kg of inner bark and wood particles were collected and discarded. After sorting, approximately 166 kg of wet outer bark particles with a size of 25 mm or more was collected.

The wet outer bark particles were dried using a hot air drier in four consecutive drying steps. After drying, approximately 94 kg of dry outer bark with a particle size of 25 mm or more was collected. Approximately 72 kg of water was collected from the drying process.

After drying, the outer bark was milled in a cutting mill to a particle size of around 20 mm which produced a mixture of dry bark with a particle size of 0-20 mm.

After milling, the particles of dry outer bark were screened using a drum screening device to remove small particles with a size of 3 mm or less. Approximately 29 kg of particles with a size of 3 mm or less were discarded and approximately 65 kg of particles with a size of 3-20 mm were collected.

Finally, the outer bark particles with a size of 3-20 mm were ground using a cutting grinder to produce approximately 65 kg of dry bark powder with a particle size of 0-0.5 mm.

### Example 2-Extraction of Betulin

120 kg (0.55 m3) of dry birch bark powder was suspended in 820 I (643 kg) of isopropanol, heated to 81-84° C. and refluxed for 4 h. The hot suspension was decanted, and the decantation liquid filtered to remove fine particles. After decantation, 250 I of isopropanol solution containing betulin and impurities was collected. 590 I of fresh isopropanol was added to the solution which was refluxed for 4 h followed by decanting to collect approximately 560 I of isopropanol solution of crude betulin.

To isolate betulin, the decanted liquids from several extraction steps were combined to form 1150 I (913 kg) of betulin solution. The solution was heated to distill of approximately 810 I of isopropanol and allowed to cool to room temperature (RT). Once the betulin suspension had cooled to RT, 280 I of isopropanol was decanted.

After decantation, the betulin was reslurried by the addition of 70 I of isopropanol and approximately 60 I of isopropanol decanted to produce a product cake made up of approximately 25 weight-% betulin, and 75 weight-% isopropanol.

The product cake was dried at 80° C. and reduced pressure (approximately 150 mbar) for 5 h. After drying, 13 kg of betulin with a purity of approximately 93% was collected. Total yield of betulin from birch bark powder was about 11% based on dry mass.

All of the isopropanol collected in the process was recycled for use in the same process.

### Example 3-Extraction of Suberin

7.5 kg (5.9 l) of isopropanol was loaded in a reactor and 0.253 kg of NaOH was added. The mixture was heated and allowed to reflux for 60 min to allow all NaOH to dissolve in the isopropanol before allowing the mixture to cool to approximately 80° C.

Approximately 3.5 kg of wet birch bark powder from Example 1 was added to the isopropanol solution and the mixture refluxed for 1.5 h while stirring before allowing the mixture to cool to about 80° C. After cooling, the formed suspension was centrifuged, and the liquid phase separated from the solid cake carefully.

The wet cake was moved to a reactor and 4.0 kg (3.14 I) of isopropanol was added. The formed mixture was refluxed for 30 min while stirring and allowed to cool to approximately 80° C. After cooling, the formed suspension was centrifuged, and the liquid phase separated from the solid cake carefully.

The combined liquid phases from both extractions were slowly cooled to 13° C. overnight.

The cooled suspension of suberin sodium salt was centrifuged and the solid cake collected. The wet suberin sodium salt was dried at 35° C. until all isopropanol had evaporated. The dried suberin sodium salt was suspended in 6 kg (6 I) of deionized water heated to 80° C. and 30% aqueous sulphuric acid added dropwise to the suspension until the pH is 2.0. The solution was stirred for 15 min to ensure that the pH was stable.

After acidifying, the suberin solution was centrifuged at 3500 rpm. The aqueous phase on top was removed and the lower emulsion phase collected.

The emulsion phase was washed four times with deionized water to produce an emulsion with a pH of over 4.3.

The emulsion was dried at 65° C. and reduced pressure to yield suberin with a purity of approximately 60% of 10-epoxy-18-hydroxyoctadecanoic acid.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A method, or an arrangement, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for processing birch bark, wherein the method comprises
providing a raw material comprising birch bark,
screening the raw material comprising birch bark to remove fines to form a screened raw material,
separating the screened raw material into a fraction comprising inner bark and wood particles, and a fraction comprising outer bark,
drying the fraction comprising outer bark to form dried bark,
cutting the dried bark in a cutting mill to form cut bark,
screening the cut bark to remove fines and form a fraction comprising bark,
milling the fraction comprising bark in a cutting milling to form a bark powder.

2. The method of claim 1, wherein the method further comprises subjecting the bark powder to a liquid-solid extraction to recover soluble components from the bark powder.

3. The method of claim 1, wherein screening the raw material comprising birch bark comprises screening the raw material comprising birch bark in a drum screening to remove particles with a particle size of 45 mm or less.

4. The method of claim 1, wherein separating the screened raw material into a fraction comprising inner bark and wood particles, and a fraction comprising outer bark comprises sorting the screened raw material optically to separate inner bark and wood particles from outer bark.

5. The method of claim 1, wherein drying the fraction comprising outer bark comprises subjecting it to at least one drying using warm air.

6. The method of claim 1, wherein the dried bark is milled in a cutting mill to a particle size of approximately 6 mm-45 mm.

7. The method of claim 1, wherein the cut bark is screened to remove particles of a size of approximately 6 mm or smaller.

8. The method of claim 1, wherein the fraction comprising bark is milled to a particle size of 0-6 mm.

9. The method of claim 1, wherein the method comprises recovering betulin and/or suberin from the bark powder.

10. The method of claim 1, wherein the method further comprises subjecting the bark powder to a liquid-solid extraction to recover betulin or suberin from the bark powder.

11. The method of claim 1, wherein the bark powder is carbonized to form birch charcoal powder.

12. An arrangement for processing birch bark, wherein the arrangement comprises
a first drum screening device,
a sorting device,
a drying device,
a first cutting mill,
a second drum screening device, and
a second cutting mill,
arranged in the above specified order.

13. The arrangement of claim 12, wherein the first drum screening device removes particles of size of 25 mm or less and the second drum screening device removes particles of a size of 3 mm or less.

14. The arrangement of claim 12, wherein the sorting device is an optical sorting device.

15. The arrangement of claim 12, wherein the drying device is a heated air drying device.

16. The arrangement of claim 12, wherein the first cutting mill cuts the material to a particle size of approximately 45 mm and the second cutting mill cuts the material to a particle size of approximately 0 mm-6 mm.
